(19) United States
(12) Patent Application Publication
(10) Pub. No.: US 2025/0073452 A1
(43) Pub. Date: Mar. 6, 2025

(54) RADIOTRANSPARENT ELECTRODE

(71) Applicant: ZOLL Medical Corporation, Chelmsford, MA (US)

(72) Inventors: Elangovan Ramanathan, Shrewsbury, MA (US); James P. Hurley, Foxborough, MA (US)

(73) Assignee: ZOLL Medical Corporation, Chelmsford, MA (US)

(21) Appl. No.: 18/821,264

(22) Filed: Aug. 30, 2024

Related U.S. Application Data
(60) Provisional application No. 63/536,160, filed on Sep. 1, 2023.

(51) Int. Cl. A61N 1/04

(52) U.S. Cl. CPC A61N 1/046; A61N 1/0496

(57) ABSTRACT

A medical system is provided. The medical system includes an electrode with a backing pad having top and bottom surfaces. A conductive layer is attached to the top surface of the backing pad, and a conductive gel layer covers at least part of the top surface of the conductive layer. A first bonding layer covers at least part of the top surface of the conductive layer. The electrode can include a leadwire with a stripped end length, and at least a portion of the stripped end length is disposed between the first bonding layer and a second bonding layer that also contacts the first bonding layer. The coupled-together first and second bonding layers are disposed at least in part between the conductive layer and the conductive gel layer.

### CROSS-REFERENCE TO RELATED APPLICATIONS

This application claims the benefit of U.S. Provisional Application No. 63/536,160, filed Sep. 1, 2023, the disclosure of which is incorporated by reference herein in its entirety.

### BACKGROUND

Cardiac arrest and other health ailments are a major cause of death worldwide. Various resuscitation efforts aim to maintain the body's circulatory and respiratory systems during cardiac arrest in an attempt to save the life of the victim. The sooner these resuscitation efforts begin, the better the victim's chances of survival. Health care professionals also attempt to detect and prevent conditions conducive to cardiac ailments by examining and treating patients. These efforts are expensive and have a limited success rate, and cardiac arrest, among other conditions, continues to claim the lives of victims.

### SUMMARY

Aspects and embodiments of the present disclosure are directed to a multifunction biomedical electrode for use during medical procedures, and systems and methods of its use and manufacture. At least one electrode can adhere to a subject to apply treatment in the form of an electric shock, for example to defibrillate, cardiovert, or pace the subject. The electrode can also pass electrical energy to the subject to stimulate a portion of the subject's body. Additionally, or alternatively, the electrode can acquire electrical signals from the subject. These signals can be used to monitor the condition of the subject. The multifunction electrode can be X-ray transmissive, for example, radiotransparent so as to exhibit complete X-ray transmission, or alternatively radiolucent so as to be substantially, but not completely, X-ray transmissive. Accordingly, an X-Ray image of a subject that is sufficient for medical diagnosis may be ascertained with the electrode adhered to the subject. In such cases, portions of the electrode may appear, for example, as a lightly shaded area in the shape of a wire on a captured X-ray image, but with sufficient transparency to see beneath the electrode into the patient during a medical procedure.

Such procedural electrodes often require the use of certain materials to provide both adequate conductivity and radio transparency. For example, the electrode may include silver/silver chloride while the leadwire coupled to the electrode may include carbon-based materials with a nickel coating to provide both sufficiently high conductivity and radio transparency. However, the use of such materials has been shown to cause problems, such as electrochemical galvanic corrosion and electrical arcing between the wire and the electrode. The corrosion and/or arcing cause damage to the electrode, lowering both its conductivity and radio transparency, and ultimately reducing shelf-life and usability of the electrode. The arcing can also be a fire hazard.

Thus, and in accordance with some embodiments, a radiotransparent electrode design is disclosed that alleviates the above-noted problems. The electrode includes a bonding layer between a stripped end of the leadwire and a conductive layer. Accordingly, the leadwire does not contact the conductive layer of the electrode. Such a design may be considered counter-intuitive as it separates the leadwire from the electrode conductive layer, where such separation may be considered to result in a decrease in the overall conductivity between the leadwire and the electrode conductive layer. However, the bonding layer is itself sufficiently conductive and mechanically rigid so as to provide a robust electrical pathway between the leadwire and the conductive layer, while also reducing or otherwise eliminating corrosion and arcing between the leadwire and the conductive layer. Accordingly, the elimination of arcing by incorporating such a bonding layer between the leadwire and conductive layer may be preferable compared to electrode designs having the leadwire directly contacting the electrode surface. In some examples, the bonding layer is a conductive tape with double-sided adhesive surfaces to bond with the conductive layer and with the stripped end of the leadwire. The double-sided conductive tape is both mechanically rigid and radio transmissive, according to some embodiments. In some examples, the bonding layer is a conductive epoxy that provides a bonding matrix between the stripped end of the leadwire and the conductive layer.

In an example, a radio transmissive electrode for providing electrotherapeutic defibrillation to a patient is provided. The electrode includes a backing pad, a conductive layer disposed upon the backing pad, a bonding layer disposed upon the conductive layer, and a leadwire comprising a stripped end length. At least a portion of the stripped end length is disposed upon the bonding layer such that the bonding layer is interposed between the conductive layer and the stripped end length.

Examples of the radio transmissive electrode may incorporate one or more of the following features.

In the radio transmissive electrode, the bonding layer is a first bonding layer and the radio transmissive electrode may further include a second bonding layer disposed on the first bonding layer and disposed on the stripped end length of the leadwire, such that the stripped end length of the leadwire is interposed between the first bonding layer and the second bonding layer. The radio transmissive electrode may further include an insulator layer disposed on the second bonding layer. The insulator layer may be disposed on at least a portion of the conductive layer. A surface area of the insulator layer may be less than half of a surface area of the backing pad. The radio transmissive electrode may further include a gel layer disposed on at least a portion of the conductive layer. The gel layer may be disposed on at least a portion of the insulator layer. The radio transmissive electrode may further include a tab coupled to a lower surface of the gel layer and protruding from the electrode. In the radio transmissive electrode, a first surface and an opposite second surface of the first bonding layer comprises an adhesive, and a first surface and an opposite second surface of the second bonding layer comprises an adhesive. The first surface of the second bonding layer may conform around the stripped end length of the leadwire.

In the radio transmissive electrode, the leadwire may include a conductive carbon element, and the stripped end length may include a plurality of stripped extensions. The plurality of stripped extensions may be in a fanned configuration. The plurality of stripped extensions may be adhesively coupled to the bonding layer.

The radio transmissive electrode may be radiolucent. The radio transmissive electrode may be radiotransparent.

The radio transmissive electrode may further include at least one of: a therapy electrode, a treatment electrode, a stimulating electrode, and a monitoring electrode, where the radio transmissive electrode is configured to adhere to a subject.

In the radio transmissive electrode, the conductive layer may include silver (Ag) and silver chloride (AgCl). The conductive layer may include more than 50% Ag by weight, and less than 50% AgCl by weight.

In the radio transmissive electrode, the bonding layer may include a plurality of sublayers. the plurality of sublayers may include a first adhesive layer, a carrier layer on the first adhesive layer, and a second adhesive layer on the carrier layer. The carrier layer may include a conductive nonwoven material.

In the radio transmissive electrode, a thickness of the bonding layer may be between about 110 micrometers and about 130 micrometers.

In the radio transmissive electrode, the bonding layer may be a conductive double-sided tape. In the radio transmissive electrode, the bonding layer may be radio transmissive. In the radio transmissive electrode, the bonding layer may be mechanically rigid. In the radio transmissive electrode, the bonding layer may be a conductive, double-sided adhesive that is radio transmissive and mechanically rigid.

In another example, a biomedical electrode system for providing electrotherapeutic defibrillation to a subject is provided. The system includes a first electrode configured to adhere to a first location of the subject, a second electrode configured to adhere to a second location of the subject, and a leadwire having a stripped end length. The first or second electrode includes a backing pad having a top surface and a bottom surface, a conductive layer attached to the top surface of the backing pad, and a bonding layer having a bottom surface attached to a portion of the conductive layer. At least a portion of the stripped end length is attached to a top surface of the bonding layer, such that the bonding layer is directly between the stripped end length of the leadwire and the conductive layer.

Examples of the biomedical electrode system may incorporate one or more of the following features.

In the system, the first electrode may be an anterior electrode configured for placement proximate to a chest of a subject, and the second electrode may be a posterior electrode configured for placement proximate to a back of the subject. The anterior electrode may have a substantially circular profile, and the posterior electrode may have a substantially rectangular profile.

In the system, the first electrode and the second electrode may be configured to form part of a circuit to apply electrical energy to a subject.

The system may further include a connector configured to receive the leadwire of the first electrode and to receive a leadwire of the second electrode.

In the system, the bonding layer is a first bonding layer and the first or second electrode may further include a second bonding layer having a bottom surface attached to the top surface of the first bonding layer and attached to the stripped end length of the leadwire, such that the stripped end length of the leadwire is directly between the first bonding layer and the second bonding layer. The system may further include an insulator layer having a bottom surface attached to a top surface of the second bonding layer. The bottom surface of the insulator layer may be attached to at least a portion of the conductive layer. A surface area of the bottom surface of the insulator layer may be less than half of a surface area of the top surface of the backing pad. The system may further include a gel layer attached to at least a portion of the conductive layer. The gel layer may be attached to at least a portion of a top surface of the insulator layer. The system may further include a tab coupled to a lower surface of the gel layer and protruding from the first or second electrode. In the system, the top and bottom surface of the first bonding layer comprises an adhesive, and a at least the bottom surface of the second bonding layer comprises an adhesive. The bottom surface of the second bonding layer may conform around the stripped end length of the leadwire.

In the system, the leadwire may include a conductive carbon element, and the stripped end length may include a plurality of stripped extensions. The plurality of stripped extensions may be in a fanned configuration. The plurality of stripped extensions may be adhesively coupled to the top surface of the bonding layer.

In the system, the first and second electrodes may be radiolucent. In the system, the first and second electrodes may be radiotransparent.

In the system, the first and second electrodes may include at least one of a therapy electrode, a treatment electrode, a stimulating electrode, and a monitoring electrode.

In the system, the conductive layer may include silver (Ag) and silver chloride (AgCl). The conductive layer may include more than 50% Ag by weight, and less than 50% AgCl by weight.

In the system, the bonding layer may be a conductive double-sided tape. In the system, the bonding layer may be radio transmissive. In the system, the bonding layer may be mechanically rigid. In the system, the bonding layer may be a conductive, double-sided adhesive that is radio transmissive and mechanically rigid.

In another example, a method of forming an electrode configured to provide electrotherapeutic defibrillation to a patient is provided. The method includes providing a backing pad having a top surface and a bottom surface; removing a first liner from a top surface of the backing pad; pressing a conductive layer onto the top surface of the backing pad; removing a second liner from a bottom surface of a first bonding layer; pressing the bottom surface of the first bonding layer onto at least a portion of the conductive layer; removing a third liner from a top surface of the first bonding layer; placing a stripped end length of a leadwire onto the top surface of the first bonding layer; removing a fourth liner from a bottom surface of a second bonding layer; and pressing the bottom surface of the second bonding layer onto the top surface of the first bonding layer and onto the stripped end length of the leadwire, such that the stripped end length of the leadwire is directly pressed between the top surface of the first bonding layer and the bottom surface of the second bonding layer.

Examples of the method may incorporate one or more of the following features.

In the method, the stripped end length of the leadwire may include a plurality of stripped extensions, and the method may further include flaring out the plurality of stripped extensions into a fanned arrangement before placing the plurality of stripped extensions onto the top surface of the first bonding layer. The method may further include applying acute pressure to a proximal end of the stripped end length of the leadwire to press the proximal end of the stripped end length down onto the top surface of the first bonding layer.

The method may further include placing the first liner over at least a top surface of the second bonding layer and pressing down on the first liner to apply compressive pressure to the first bonding layer and the second bonding layer.

The method may further include removing a fifth liner from a top surface of the second bonding layer and placing an insulator layer on at least the top surface of the second bonding layer. Placing the insulator layer may include placing the insulator layer on at least a first portion of the conductive layer. The method may further include placing a gel layer on at least a second portion of the conductive layer different from the first portion. Placing the gel layer may include placing the gel layer on at least a portion of the insulator layer.

Still other aspects, examples, and advantages of these aspects and examples, are discussed in detail below. Moreover, it is to be understood that both the foregoing information and the following detailed description are merely illustrative examples of various aspects and features and are intended to provide an overview or framework for understanding the nature and character of the claimed aspects and examples. Any example or feature disclosed herein can be combined with any other example or feature. References to different examples are not necessarily mutually exclusive and are intended to indicate that a particular feature, structure, or characteristic described in connection with the example can be included in at least one example. Thus, terms like “other” and “another” when referring to the examples described herein are not intended to communicate any sort of exclusivity or grouping of features but rather are included to promote readability.

The systems and methods described herein are not limited in their application to the details of construction and the arrangement of components set forth in the description or illustrated in the drawings. The innovations described herein are capable of other embodiments and of being practiced or of being carried out in various ways. Also, the phraseology and terminology used herein is for the purpose of description and should not be regarded as limiting. The use of “including” “comprising” “having” “containing” “involving” and variations thereof herein, is meant to encompass the items listed thereafter, equivalents thereof, and additional items, as well as alternate embodiments consisting of the items listed thereafter exclusively.

### BRIEF DESCRIPTION OF THE DRAWINGS

Various aspects of the disclosure are discussed below with reference to the accompanying figures, which are not intended to be drawn to scale. The figures are included to provide an illustration and a further understanding of various examples and are incorporated in and constitute a part of this specification but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure. A quantity of each component in a particular figure is an example only and other quantities of each, or any, component could be used.

FIG. 1A illustrates a plan view of an electrode, in accordance with an embodiment of the present disclosure.

FIG. 1B illustrates an exploded perspective view of the electrode of FIG. 1A.

FIG. 1C illustrates a plan view of another electrode, in accordance with an embodiment of the present disclosure.

FIG. 1D illustrates an exploded perspective view of the electrode of FIG. 1C.

FIG. 1E illustrates a layer structure of a bonding layer in an electrode, in accordance with some embodiments of the present disclosure.

FIGS. 2A-2I illustrate various stages in the fabrication of an electrode, in accordance with some embodiments of the present disclosure.

FIG. 3 is a flow diagram depicting a process for fabricating an electrode, in accordance with some embodiments of the present disclosure.

FIG. 4A illustrates a plan view of a top surface of an electrode label that may be used with an electrode, in accordance with an embodiment of the present disclosure.

FIG. 4B illustrates a plan view of a top surface of an electrode label that may be used with an electrode, in accordance with an embodiment of the present disclosure.

FIG. 5A illustrates a plan view of a top surface of an electrode label that may be used with an electrode, in accordance with an embodiment of the present disclosure.

FIG. 5B illustrates a plan view of a top surface of an electrode label that may be used with an electrode, in accordance with an embodiment of the present disclosure.

FIG. 6 illustrates a plan view of a pair of electrodes as part of a biomedical electrode system in accordance with some embodiments of the present disclosure.

FIG. 7 illustrates a plan view of a pair of electrodes as part of a biomedical electrode system in accordance with some embodiments of the present disclosure.

### DETAILED DESCRIPTION

During a surgical procedure, time is typically of the essence if and when emergency care is needed for the subject. Often, the subject may have one or more incisions made to their chest or thorax area when performing any procedure, for example, related to their heart or other surrounding vital organs. During any such procedure, the subject may experience sudden cardiac arrest or any other cardiac emergency that requires swift medical attention. However, it can be difficult to place electrodes needed for defibrillation after the operation has begun due to the location of any incisions and/or other medical devices on or near the subject. For example, the subject may have a 12 lead electrode arrangement placed on their torso for electrocardiogram (ECG) analysis, which can interfere with later placement of defibrillation electrodes. Such procedures can be especially difficult during a chaotic situation in which medical personnel may be scrambling to save the life of the subject experiencing the medical emergency during an operation.

For at least the reasons discussed above, as a precautionary measure, defibrillation electrodes may be placed on the subject before an operation is performed where it may be possible that the subject may experience a cardiac arrest during the procedure. In this way, the electrodes are already in place in case a medical emergency arises during the operation. However, common electrode materials (such as copper or other similar metals) block x-rays or other imaging waves, which can make it difficult for the medical personnel to view into the body during an operation. Thus, and in accordance with some embodiments of the present disclosure, the defibrillation electrodes are made using radio transmissive material. A material that is radio transmissive can be either radiolucent (partially transparent to x-rays) or radiotransparent (fully transparent to x-rays), such as a silver/silver chloride electrode surface and carbon-based wires. A material that is radiolucent may allow at least 95%, at least 90%, at least 80%, at least 75%, or at least 50% of the x-ray energy to pass through it.

Defibrillator electrodes may remain in storage for an extended period of time before they are used. In some examples, defibrillator electrodes may remain stored in hospitals, ambulances, operation rooms, or other medical facilities for several years before use. Such extended storage can degrade electrodes over time, due to effects such as galvanic corrosion. In some examples, the metal coating used around the wires (e.g., a nickel coating) that contacts the electrode surface can lead to corrosion in conjunction with the conductive material of the electrode surface. According to some embodiments, separating the direct contact between the exposed conductive wires and the conductive electrode surface can reduce or eliminate corrosion damage to the wires and/or electrode, as will be discussed in more detail herein.

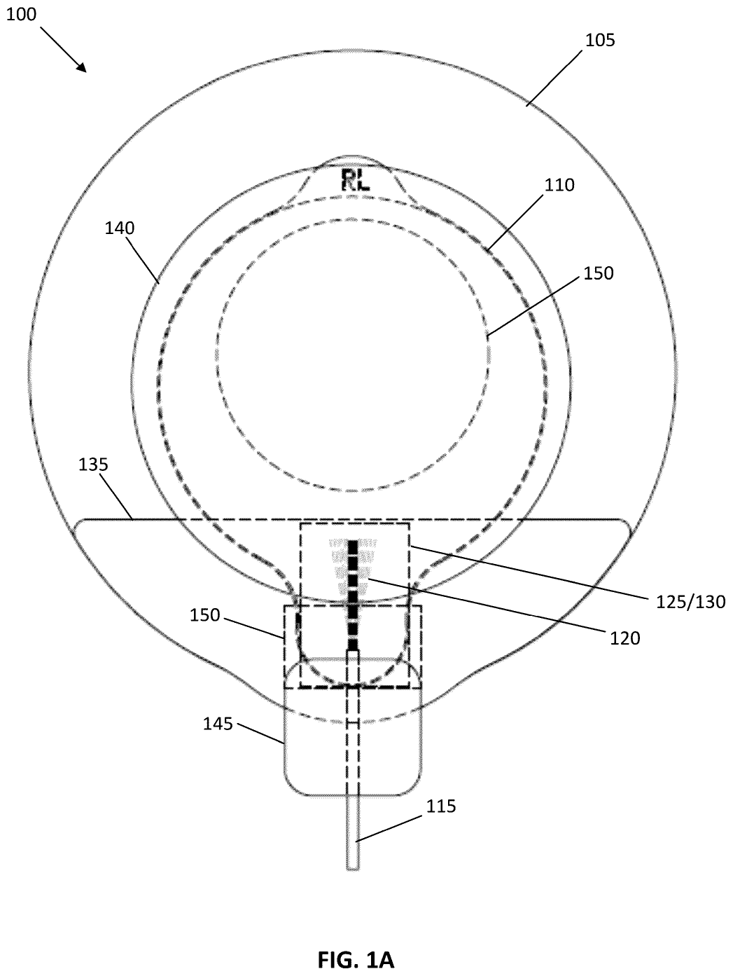

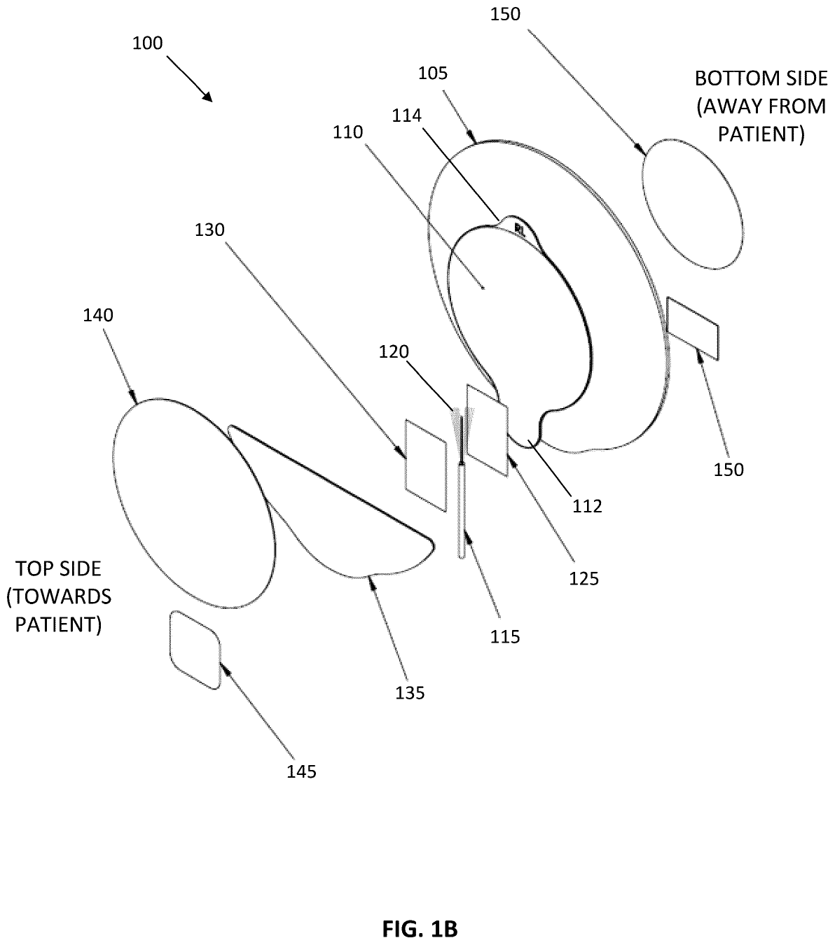

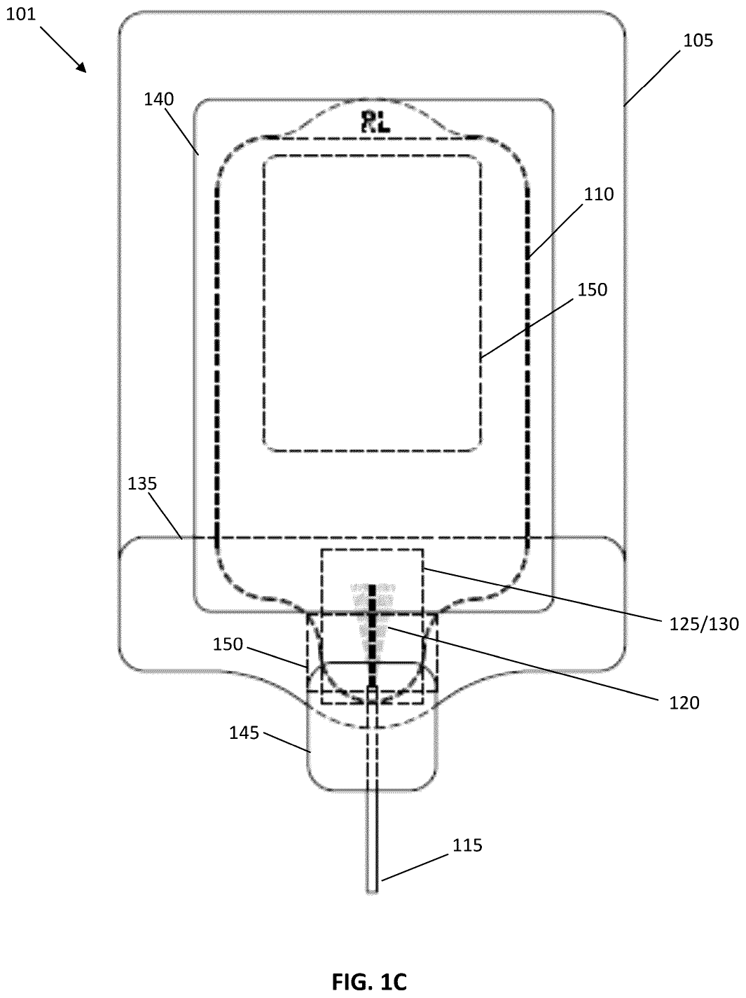

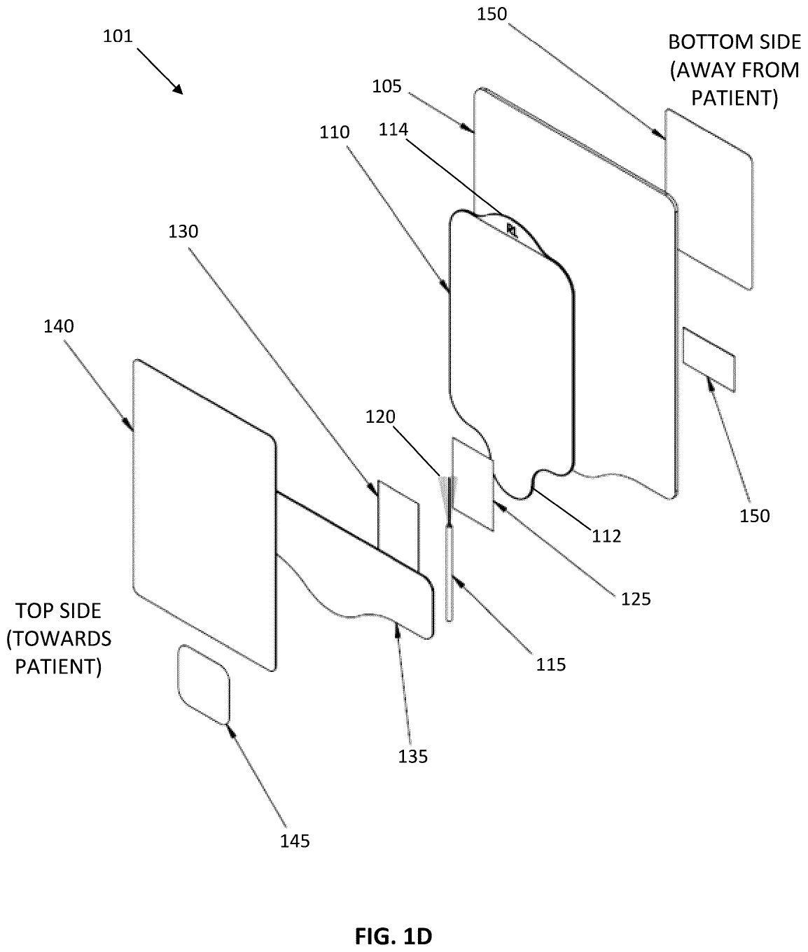

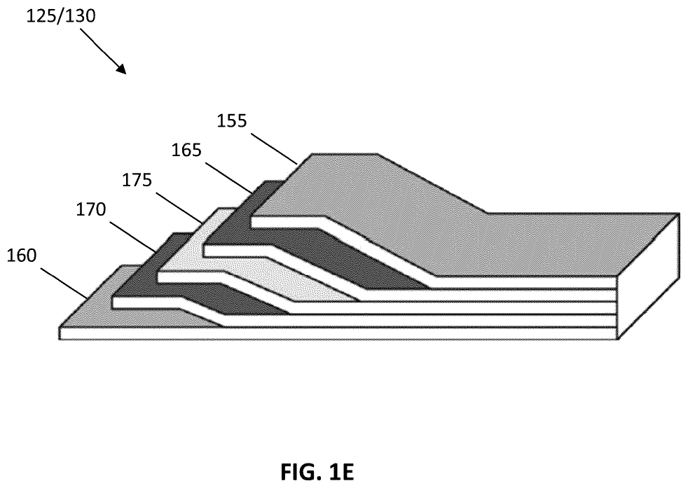

FIG. 1A is a plan view depicting an electrode 100 in accordance with one embodiment, and FIG. 1B is an exploded view of electrode 100 of FIG. 1. FIG. 1C is a plan view depicting another electrode 101 in accordance with another embodiment, and FIG. 1D is an exploded view of electrode 101 of FIG. 1C. According to some embodiments, electrodes 100 and 101 may have similar layer structures and materials but differ in cross-sectional shape. In the illustrated examples, electrode 100 has a generally circular cross-section while electrode 101 has a generally rectangular cross-section. For ease of discussion, the same components from each of electrode 100 and electrode 101 are provided the same label. It should be understood that any discussion herein regarding electrode 100 applies equally to electrode 101, unless stated otherwise.

In an embodiment, electrode 100 is a multifunction procedural electrode. For example, electrode 100 can be a biomedical electrode to administer an electric shock to a subject for defibrillation, pacing, or to stimulate muscle contraction. Electrode 100 can also be a monitoring electrode to monitor or detect, for example, electrical activity of a subject's heart. This information can be used to generate an electrocardiogram. Electrode 100 can be placed proximate to a subject and coupled with power sources and control logic to deliver electrical energy to the subject, to determine the timing, levels, and history of applied energy, and to process monitored or detected data for analysis by a health care provider, for example. Electrode 100 may be located proximate to the subject, e.g., attached, connected, or coupled to the subject, at an anterior, posterior, lateral, or other location of the subject. For example, electrode 100 can be attached to the subject's chest, back, side, head, abdomen, torso, thorax, or legs. In one embodiment, electrode 100 is an external electrode attached to the subject proximate to the subject's heart. Electrode 100 can be disposable or configured for repeated use.

Electrode 100 has a top side and an opposite bottom side. In one embodiment, when proximate to a subject, the bottom side of electrode 100 and its components face away from the subject, and the top side of electrode 100 and its components face toward the subject. FIGS. 1B and 1D identify which surfaces of electrode 100/101 are considered the top side and the bottom side, according to an embodiment. Additionally, each element of electrode 100 may have a top surface that faces the top side and an opposite bottom surface that faces the bottom side. Some of the top surfaces of the various elements of electrode 100 may contact the subject by, for example, adhering to the subject's skin. In one embodiment, electrode 100 is substantially flat. For example, electrode 100 may have a flat profile that is not noticeable or is minimally noticeable when attached to the subject, under the subject's clothes. Electrode 100 may also be substantially flexible. For example, electrode 100 can conform to the contours of the subject's body during initial attachment to the subject, and can conform to body positioning changes when the subject is in motion. Electrode 100 can also be substantially devoid of rigid components, such as hard snaps, connectors, and rigid plates. For example, electrode 100 may be devoid of hard rigid substances that may cause uncomfortable pressure points when a subject with electrode 100 attached to his/her body is in a prone, prostrate, supine, or lateral position with electrode 100 pressed against an object, such as a bed, couch, medical examining table, clothes, or medical equipment. Electrode 100 can be compliant with the American National Standards Institute (ANSI) standards for electrodes, or with the Association for the Advancement of Medical Instrumentation (AAMI) standards for electrodes.

In one embodiment, electrode 100 includes at least one backing pad 105, with a bottom surface of backing pad 105 configured to face away (e.g., outward) from the subject wearing electrode 100 and a top surface of backing pad 105 configured to face toward (e.g., inward) the subject. In one embodiment, backing pad 105 includes a flexible, stretchable film, such as polyethylene foam that insulates the bottom (e.g., outer) surface of electrode 100 from electrical current. In one embodiment, backing pad 105 has a thickness of 1/16^{th} of an inch, +/−20%. Backing pad 105 can have other thicknesses, both more and less than 1/16^{th} of an inch. Backing pad 105 may include instructions, pictures, identifiers, or warnings. For example, words, symbols, or images can be printed on the top or bottom surfaces of backing pad 105.

According to an embodiment, the top surface of backing pad 105 includes an adhesive, such as a pressure sensitive adhesive. The adhesive may cover all, substantially all (e.g., at least 90%), or part of the top surface of backing pad 105 to provide adhesion of electrode 100 to the subject or to other elements of electrode 100. The bottom surface of backing pad 105 may be free of adhesive. In one embodiment, backing pad 105 defines the footprint, or outer boundary or profile of electrode 100. Portions of electrode 100 components (e.g., wires, tabs, labels) may extend beyond this footprint.

Backing pad 105 may have various shapes and sizes that delineate the footprint of electrode 100. For example, backing pad 105 and electrode 100 can be substantially circular, as depicted in FIGS. 1A and 1B. A substantially circular shape may be a circular shape, an oval shape, or a circular shape with one or more protruding sides. A circular backing pad 105 may have a diameter of between 5.5 and 6.5 inches. Backing pad 105 can have other diameters above and below this range, such as between 3 and 4 inches, or between 2 and 3 inches. In one embodiment, a circular backing pad 105 is part of an anterior electrode 100 configured for placement proximate to the subject's chest. In one embodiment, backing pad 105 and electrode 101 are rectangular, as depicted in FIGS. 1C and 1D. A substantially rectangular shape may be a rectangular shape with sharp corners or a rectangular shape with rounded corners. A substantially rectangular backing pad 105 may have a length of between 6 and 7 inches (e.g., 6.5 inches), and a width of between 4.5 and 5.5 inches (e.g., 5 inches). In some embodiments, backing pad 105 is substantially rectangular with a length of between 4.5 and 5.5 inches and a width between 3 and 4 inches, or a length between 2 and 3 inches and a width between 1.5 and 2.5 inches. Backing pad 105 may also have other dimensions, greater and less than these ranges, as well as larger and smaller length to width ratios. In one embodiment, a substantially rectangular backing pad 105 is part of a posterior electrode 101 configured for placement proximate to the subject's back.

In an embodiment, electrode 100 includes at least one conductive layer 110. Electrical energy can transfer from a power source coupled with electrode 100 to a subject wearing electrode 100 via conductive layer 110. For example, conductive layer 110 can conduct sufficient electrical current (e.g., up to 60 Amps for up to 40 milliseconds) over a sufficiently large area to apply defibrillation or pacing therapy to the subject, and with sufficient energy (e.g., 1-370 Joules). In one embodiment, conductive layer 110 includes a film covered at least in part with conductive ink. The film that is covered with the conductive ink may be conductive or non-conductive. The conductive ink can be applied to the film via, for example, a screen printing method. In one embodiment, conductive layer 110 includes a metal/metal chloride covering, such as silver/silver chloride. In some embodiments, conductive layer 110 includes over 50%, over 60%, over 70%, over 80%, or over 90% silver by weight and less than 50%, less than 40%, less than 30%, less than 20%, or less than 10% silver chloride by weight. Conductive layer 110 may be flexible (e.g., a film covered with conductive ink) or rigid (e.g., a metal or other conductive plate). In one embodiment, conductive layer 110 includes a non-conductive plate covered at least in part with conductive ink.

In one embodiment, a bottom surface of conductive layer 110, which faces away or outward from a patient wearing electrode 100, is coupled with the top surface of backing pad 105. For example, the bottom surface of conductive layer 110 or the top surface of backing pad 105 can include an adhesive to couple these two elements together. Conductive layer 110 may be substantially circular, for example as depicted in FIGS. 1A and 1B. A substantially circular conductive layer 110 may have a diameter of substantially 3.5 inches, (e.g., between 3 and 4 inches), or substantially 2 inches (e.g., between 1.5 and 2.5 inches). A substantially circular conductive layer can have other diameters greater and less than these ranges. In some embodiments, conductive layer 110 is substantially rectangular, for example as depicted in FIGS. 1C and 1D. A rectangular conductive layer 110 can have a length of substantially 4.5 inches (e.g., 4 to 5 inches) and a width of substantially 3 inches (e.g., 2.5 to 3.5 inches). In some embodiments, rectangular conductive layer 110 has a length of substantially 3.25 inches (e.g., between 2.5 and 3.5 inches) and a width of substantially 1.75 inches (e.g., between 1.5 and 2.5 inches); or a length of substantially 2.0 inches (e.g., between 1.5 and 2.5 inches) and a width of substantially 1.75 inches (e.g., between 1.0 and 2.0 inches).

Conductive layer 110 in circular, square, or other configurations may also include at least one protruding tab or extension that extends approximately one inch out from the edges of conductive layer 110, (e.g., deviating from the circumference of the circle or parallel line outline of the rectangle). Components of electrode 100 can couple to this protrusion to connect with conductive layer 110. In one embodiment, conductive layer 110 includes two protrusions, a first protrusion 112 of approximately one inch that may connect with components of electrode 100, and a second protrusion 114 that may be smaller than the first protrusion and that may contain information about electrode 100 or about conductive layer 110.

In one embodiment, conductive layer 110 is made up of a radio transmissive material and protrusion 114 may include writing or symbols to indicate, for example, that conductive layer 110 is radio transmissive in nature, or to identify top or bottom surfaces to assist and verify the assembly or disassembly processes. Conductive layer 110, for example, includes at least some degree of X-Ray transmissivity, with minimal or generally uniform X-Ray attenuation. In some embodiments, conductive layer 110 may be made up of a material that at least partially appears in an X-Ray image, however, the X-Ray image of the portion of the subject's body covered by conductive layer 110 can be of sufficient quality to allow for a medical diagnosis. Accordingly, the subject can continuously wear electrode 100, including during periods of medical examination, without hindering the examination. In some cases, conductive layer 110 may be completely radio transmissive and may not appear or be otherwise detectable by inspection on any X-Ray image.

In one embodiment, electrode 100 includes at least one leadwire 115. For example, one end of leadwire 115 can couple with electrode 100, and another end of leadwire 115 can couple with a power source. In one embodiment, electrode 100 including leadwire 115 form part of a circuit that includes the subject, and electrical current can be applied to the subject wearing electrode 100 from the power source via leadwire 115. The power source may be associated with a defibrillator, pacing unit, or monitor. Electrical signals sensed by electrode 100 can also be provided from electrode 100 to a control unit of a defibrillator, pacing unit, or monitor for processing or evaluation. In one embodiment, leadwire 115 has a length of at least 36 inches, for example, up to around 120 inches. The leadwire can also have a length of less than 36 inches.

Leadwire 115 may include a low resistance wire, for example having a resistance of less than 0.2 Ohms/foot, with an insulated coating or jacket. In one embodiment, leadwire 115 includes metal or conductive metal coated carbon fibers. For example, leadwire can include a 12,000 strand nickel plated carbon wire fiber with up to 4 tinned copper strands. In one embodiment, leadwire 115 includes a stripped end length 120. Stripped end length 120 includes an unsheathed portion of leadwire 115 with exposed conductive wires or filaments. Stripped end length 120 may have a length of substantially one inch or less. For example, stripped end length 120 may include a plurality of extensions (e.g., wires, strands, or filaments) extending in a fanned configuration, with each extension having a length of one inch or less. The lengths of the individual extensions can be uniform or they may vary. Stripped end length 120 can also have a length greater than one inch. In one embodiment, stripped end length 120 does not interfere with radio transmissive characteristics of electrode 100, e.g., a medical diagnosis may be ascertained with stripped end length 120 being at least partially visible in an X-Ray image of a subject.

The individual extensions of stripped end length 120 can have various cross-sectional shapes. For example, the extensions may have a substantially round cross section. In one embodiment, at least one extension of stripped end length 120 is substantially flat. For example, a flattened extension may have at least two flat surfaces, such as a top surface (e.g., facing the top side of the electrode) and a bottom surface (e.g., facing the bottom side of the electrode). These two surfaces can be substantially parallel. In one embodiment, stripped end length 120 and leadwire 115 are made up of materials that are partially or completely radio transmissive.

According to some embodiments, electrode 100 includes a first bonding layer 125 disposed between stripped end length 120 and conductive layer 110. In this configuration, no part of stripped end length 120 directly contacts conductive layer 110. According to an embodiment, first bonding layer 125 includes a low elongation, high tensile strength film, such as 0.0005 inch thick polyester. First bonding layer 125 contributes rigidity to electrode 100 that helps to reduce or prevent bending of stripped end length 120 that may cause stripped end length 120 to break or separate from first bonding layer 125. In some embodiments, first bonding layer 125 is made up of a radio transmissive material. The top surface and the bottom surface of first bonding layer 125 may each include an adhesive to couple first bonding layer 125 with proximate electrode 100 components, (e.g., coupling a top surface of first bonding layer 125 to stripped end length 120 and coupling a bottom surface of first bonding layer 125 to conductive layer 110). The adhesive used on either surface can be conductive or non-conductive, pressure sensitive, epoxy, or cyanoacrylate. According to some embodiments, the adhesiveness of one or both of the top surface and the bottom surface of first bonding layer 125 provides enhanced rigidity against shear forces that may otherwise delaminate first bonding layer 125 from an adjacent electrode element or cause movement of first bonding layer 125 across an adjacent electrode element. In one embodiment, the adhesive on the top surface of first bonding layer 125 couples first bonding layer 125 with stripped end length 120, holding stripped end length 120 in place upon the top surface of first bonding layer 125, and the adhesive on the bottom surface of first bonding layer 125 couples first bonding layer 125 to first protrusion 112 of conductive layer 110. In some embodiments, a portion of leadwire (e.g., a portion of the insulative jacket around the conductive wires) 115 is also coupled to a portion of the top surface of first bonding layer 125.

According to some embodiments, electrode 100 includes a second bonding layer 130 disposed over stripped end length 120, such that stripped end length 120 is sandwiched between first bonding layer 125 and second bonding layer 130. Second bonding layer 130 may similarly include a low elongation, high tensile strength film, such as 0.0005 inch thick polyester. The top surface and the bottom surface of second bonding layer 130 may each include an adhesive to couple second bonding layer 130 with proximate electrode 100 components, (e.g., coupling a top surface of second bonding layer 130 to an insulator layer 135 and coupling a bottom surface of second bonding layer 130 to a top surface of first bonding layer 125 and to stripped end length 120). The adhesive used on either surface can be conductive or non-conductive, pressure sensitive, epoxy, or cyanoacrylate. In one embodiment, the adhesive on the bottom surface of second bonding layer 130 couples second bonding layer 130 with stripped end length 120, holding stripped end length 120 in place between the top surface of first bonding layer 125 and the bottom surface of second bonding layer 130. In some embodiments, a portion of leadwire 115 (e.g., a portion of the insulative jacket around the conductive wires) is also coupled to a portion of the bottom surface of second bonding layer 130.

In some embodiments, either one of or both first bonding layer 125 and second bonding layer 130 is a pressure sensitive adhesive, such as a conductive double-sided tape. FIG. 1E illustrates a view of the various sublayers that can make up first or second bonding layer 125/130. According to some embodiments, bonding layer 125/130 includes a first liner layer 155 on a top surface of bonding layer 125/130, a second liner layer 160 on a bottom surface of bonding layer 125/130, a first adhesive layer 165, a second adhesive layer 170, and a carrier layer 175 between first adhesive layer 165 and second adhesive layer 170. As noted above, the adhesiveness of both first adhesive layer 165 and second adhesive layer 170 is sufficient to provide enhanced rigidity against shear forces that may otherwise delaminate one of or both first bonding layer 125 and second bonding layer 130 from an adjacent electrode element or cause movement of one of or both first bonding layer 125 and second bonding layer 130 across an adjacent electrode element.

Each of first liner layer 155 and second liner layer 160 may be removed to expose the adjacent first adhesive layer 165 or second adhesive layer 170, respectively. According to some embodiments, both liner layers are removed when assembling electrode 100, such that first bonding layer 125 and second bonding layer 130 each include only first adhesive layer 165, second adhesive layer 170, and carrier layer 175.

Each of first adhesive layer 165 and second adhesive layer 170 may be substantially similar, each with a thickness between about 40 micrometers and about 60 micrometers. Each of first adhesive layer 165 and second adhesive layer 170 may include conductive materials such as graphite, silver particles, nickel particles, or silver coated nickel particles. Carrier layer 175 may have a thickness between about 10 micrometers and about 30 micrometers. According to some embodiments, carrier layer 175 includes a conductive nonwoven material, such as a nonwoven polyester that is coated with a thin layer of nickel or copper. The combination of first adhesive layer 165, second adhesive layer 170, and carrier layer 175 may exhibit a volume resistance of less than about 10 Ohms and a surface resistance of less than about 15 Ohms.

According to some embodiments, either one of or both first bonding layer 125 and second bonding layer 130 is a conductive epoxy. The conductive epoxy may include a conductive filler, such as silver, nickel, graphite, or silver coated nickel. Epoxies with silver filler may achieve a relatively low resistivity, such as less than 0.01 Ohm-cm or less than 0.001 Ohm-cm. Epoxies with a graphite filler may have a relatively higher resistivity, such as 5-10 ohm-cm or 20-40 ohm-cm, but may also exhibit a higher radiolucency compared to epoxies with the silver filler.

According to some embodiments, first bonding layer 125 includes a conductive pressure sensitive adhesive, such as described in FIG. 1E, or a conductive epoxy and second bonding layer 130 includes a non-conductive pressure sensitive adhesive or a non-conductive epoxy.

According to an embodiment, electrode 100 includes at least one insulator layer 135. Insulator layer 135 may include a non-conductive layer of flexible, stretchable film, such as polyethylene foam. While the thickness of insulator layer 135 may vary, in one embodiment, insulator layer 135 is 1/32nd of an inch thick. The top surface and/or the bottom surface of insulator layer 135 may include an adhesive, such as a pressure sensitive adhesive. Insulator layer 135 can insulate the subject from current that may be present at stripped end length 120. In one embodiment, insulator layer 135 is disposed between the subject and stripped end length 120 so that the subject does not directly contact stripped end length 120 or any part of first bonding layer 125 or second bonding layer 130.

In one embodiment, at least a portion of the top surface of insulator layer 135 contacts the subject. The bottom surface of insulator layer 135 may contact portions of at least one of first bonding layer 125, second bonding layer 130, conductive layer 110, and backing pad 105. The bottom surface of insulator layer 135 may also contact portions of leadwire 115 (e.g., a portion of the insulative jacket around the conductive wires) or stripped end length 120. Second bonding layer 130 may be included as part of insulator layer 135, or they may be distinct elements. The outer boundary of insulator layer 135 may follow a portion of the footprint of electrode 100 delineated by backing pad 105, without extending beyond this footprint. In one embodiment, the surface area of insulator layer 135 (top or bottom surface) is less than one third or less than half the surface area of backing pad 105 (top or bottom surface).

Electrode 100 may also include at least one conductive gel layer 140. Conductive gel layer 140 generally provides an interface between conductive layer 110 and the subject's skin to deliver current to, and receive signals from the subject's body. In one embodiment, conductive gel layer 140 is a conductive gel pad. The bottom surface of conductive gel layer 140 can include an adhesive to couple with proximate electrode 100 components (e.g., insulator layer 135 and conductive layer 110). The top surface of conductive gel layer 140 may be designed to attach to the subject wearing electrode 100. Conductive gel layer 140 may include a conductive adhesive polymer hydrogel, gel pad, gel sponge, or conductive fluid. Conductive gel layer 140 may include gel disposed in a membrane. The top and bottom surfaces of the membrane can include an adhesive. In one embodiment, conductive gel layer 140 includes conductive fluid in a membrane, and the passing of current through electrode 100 to the subject may release at least some of the conductive fluid from the membrane, for example by rupturing the membrane.

In one embodiment, conductive gel layer 140 covers the bottom surface of conductive layer 110. Conductive gel layer 140 can cover more than the complete bottom surface of conductive layer 110. For example, conductive gel layer 140 can extend at least ¼ inch beyond the edges (e.g. footprint) of conductive layer 110. In one embodiment, conductive gel layer 140 directly contacts conductive layer 110 and extends at least ⅛ inch beyond the edges of conductive layer 110. Intervening elements, such as insulator layer 135, first bonding layer 125, second bonding layer 130, or stripped end length 120 may be present between the bottom surface of conductive gel layer 140 and the top surface of conductive layer 110. For example, the bottom surface of conductive gel layer 140 can couple with any one or more of the top surface of insulator layer 135, the top surface of first bonding layer 125, the top surface of second bonding layer 130, stripped end length 120, leadwire 115, the top surface of conductive layer 110, or the top surface of backing pad 105. In one embodiment, conductive gel layer 140 has a similar shape but different size than electrode 100. For example, each of backing pad 105 and conductive layer 110 of electrode 100 may have a circular shape and include a circular conductive gel layer 140 (as shown in FIGS. 1A and 1B), or a rectangular shape and a rectangular conductive gel layer 140 (as shown in FIGS. 1C and 1D).

Conductive gel layer 140 may have at least one of an area of at least 12.6 in², or a diameter of substantially 4 inches (e.g., 3.5 to 4.5 inches). In some embodiments, conductive gel layer 140 has an area of at least 6.5 in², or a diameter of substantially 3 inches (e.g., 2.5 to 3.5 inches), or an area of at least 4.0 in², or a diameter of substantially 2.25 inches (e.g., 2.0 to 3.0 inches). In one embodiment, conductive gel layer 140 is rectangular shaped with a length of substantially 5 inches (e.g., 4.5 to 5.5 inches) and a width of substantially 3.5 inches (e.g., 3.0 to 4.0 inches). In some embodiments, conductive gel layer 140 is rectangular shaped with a length of substantially 3.75 inches (e.g., 3.25 to 4.25 inches) and a width of substantially 2.25 inches (e.g., 1.75 to 2.75 inches), or with a length of substantially 2.25 inches (e.g., 1.75 to 2.75 inches) and a width of substantially 1.75 inches (e.g., 1.25 to 2.25 inches). In one embodiment, at least part of the edge of backing pad 105 extends at least a half inch beyond the edge of conductive gel layer 140. In one embodiment, conductive gel layer 140 or electrode 100 and its components are in compliance with the requirements of the ANSI or AAMI DF80 standards for electrodes. Protruding tabs or extensions that may protrude out from the edges of conductive layer 110 may extend beyond the footprint of conductive gel layer 140.

Electrode 100 may also include at least one tab 145. In one embodiment, tab 145 is a plastic sheet protruding from electrode 100 that the subject or health care provider may use as a handle to remove electrode 100 from the subject or from its packaging. Tab 145 can couple to any part of electrode 100, such as the top surface of insulator layer 135. In some examples, the top surface of tab 145 contacts the subject. In one embodiment, the top surface of tab 145 and the portion of the bottom surface of tab 145 that protrudes out from backing pad 105 are free of adhesive. In one embodiment, tab 145 identifies itself as a tab to be used for gripping when adjusting, placing, moving, or removing electrode 100. For example, tab 145 may include written instructions, symbols, or may be brightly colored (e.g., red) to indicate that it may operate as a handle or to be pulled to delaminate electrode 100 from the skin of the subject. In one embodiment, tab 145 is square shaped having dimensions of 1.25×1.25 inches or less.

According to some embodiments, electrode 100 includes at least one label 150. Labels 150 can be disposed on the bottom side of backing pad 105, or can protrude from electrode 100. In one embodiment, labels 150 include printed instructions (e.g., words, pictures, or symbols) for the operation, placement, or removal of electrode 100 from the subject. Labels 150 may be made from a flexible printable film and coupled to electrode 100 with a pressure sensitive adhesive. Labels 150 can have a similar shape as electrode 100 (e.g., circular, rectangular) and can be smaller than backing pad 105. Labels 150 can be separate elements attached to electrode 100, or can be printed directly on electrode components, such as the bottom surface of backing pad 105.

Electrode components may adhere to adjacent components by use of adhesive on their surfaces, and one surface of one element can adhere to more than one adjacent component. For example, the top surface of backing pad 105 can contact the bottom surfaces of conductive layer 110, first bonding layer 125, insulator layer 135, and conductive gel layer 140. The subject wearing electrode 100 can also contact these and other components. The surfaces of electrode components may, but need not, cover the entire surfaces of their adjacent components.

In one embodiment, electrode components being disposed on, proximate to, coupled with, connected to, attached to, or located between any electrode component includes the components being at least partially disposed on, proximate to, coupled with, connected to, attached to, or located between any electrode component. Intervening components may be present.

### Electrode Fabrication

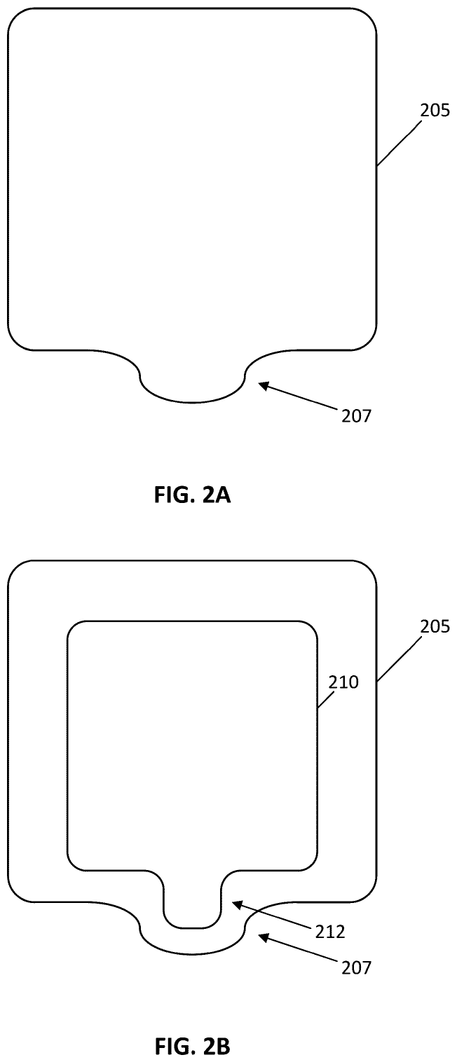

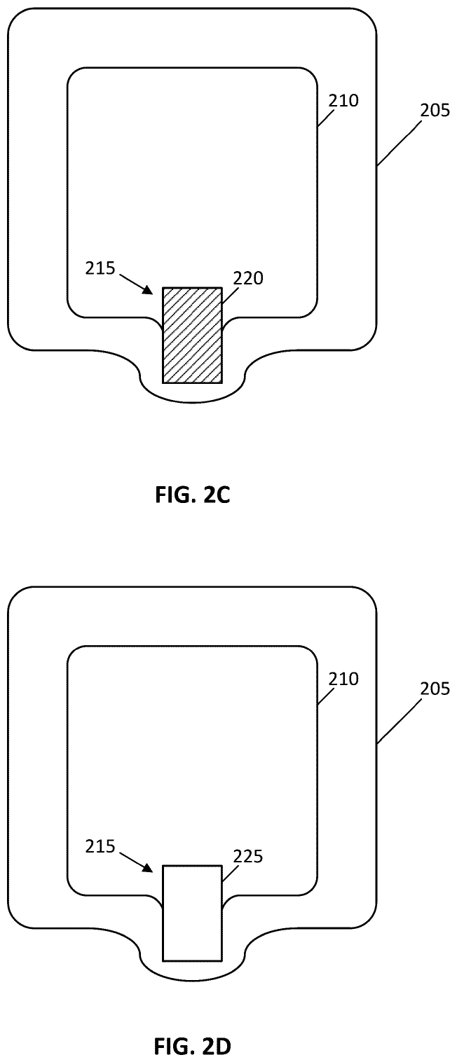

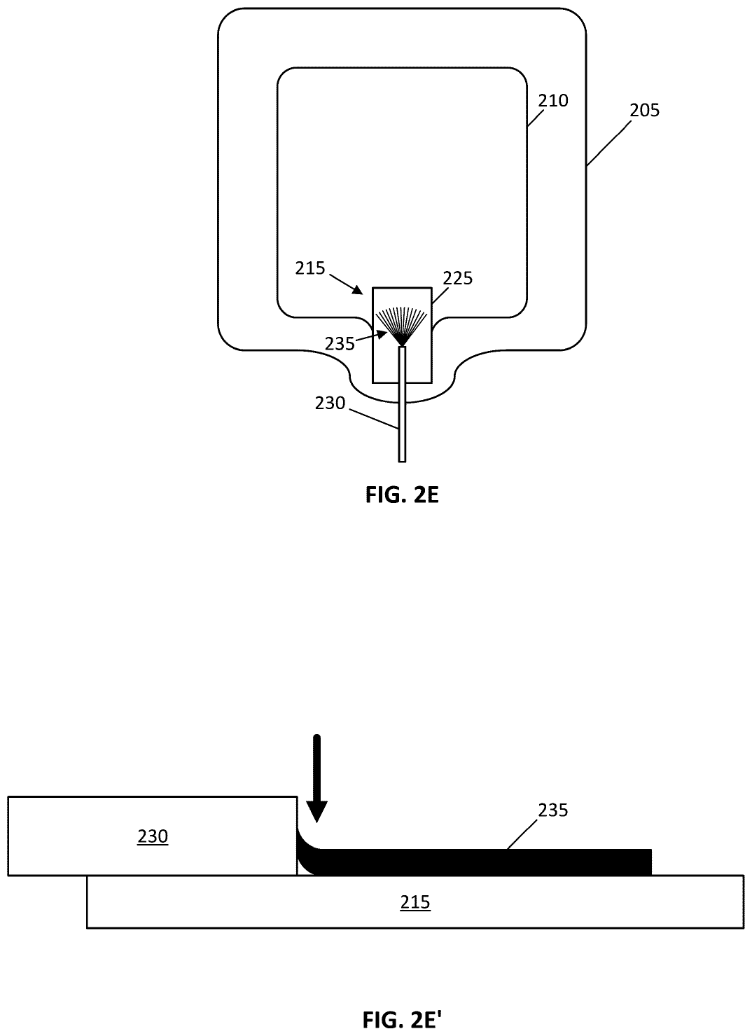

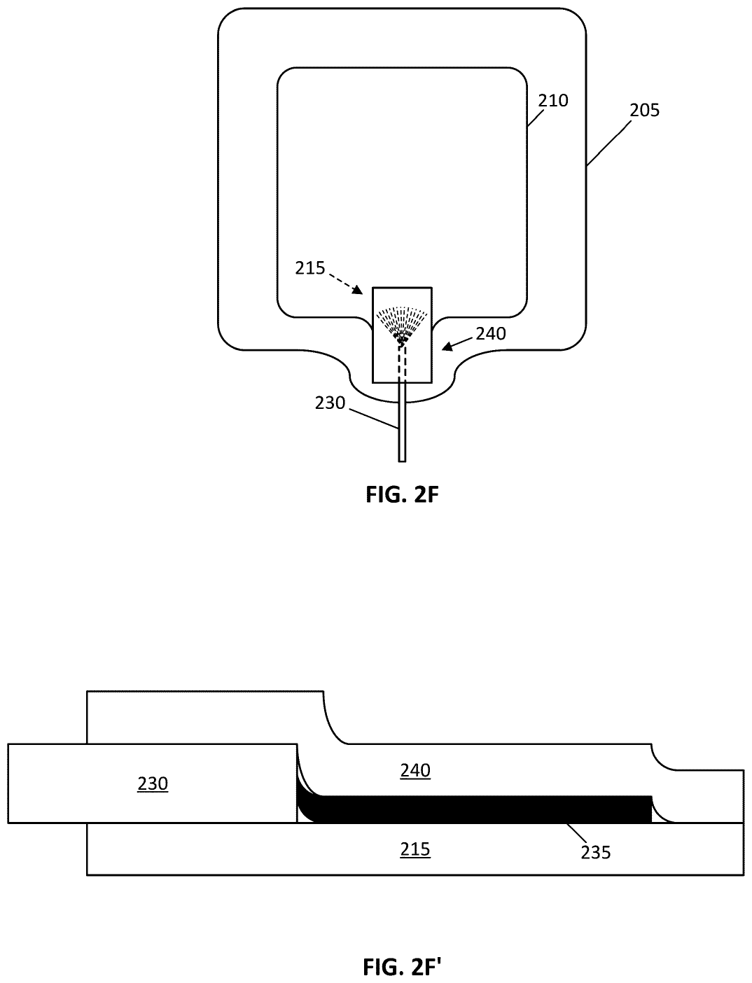

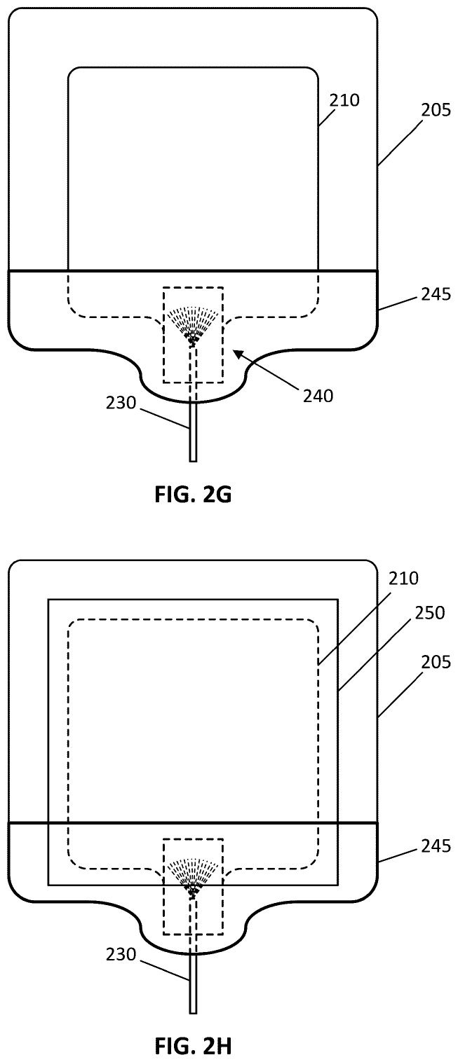

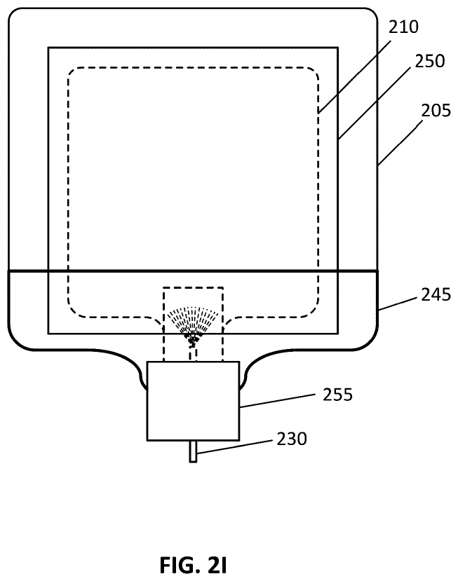

FIGS. 2A-2I include different views that collectively illustrate an example process for forming an electrode, in accordance with an embodiment of the present disclosure. Each figure shows an example structure that results from the process flow up to that point in time, so the depicted structure evolves as the process flow continues, culminating in the structure shown in FIG. 2I. FIGS. 2A-2I illustrate a rectangular electrode, however, the depicted operations could be used to form an electrode of any shape, such as a circular electrode. Example materials and process parameters are given, but the present disclosure is not intended to be limited to any specific such materials or parameters, as will be appreciated. With regards to each of the electrode elements, surfaces that face upwards (away from the page) are facing towards the top side of the electrode and surfaces that face downwards (into the page) are facing towards the bottom side of the electrode.

FIG. 2A illustrates the beginning of the electrode fabrication using a backing pad 205. As discussed above, backing pad 205 may be a foam-based material that includes an adhesive top surface that faces upwards (away from the page). In some examples, a first liner over the adhesive top surface is removed to expose the adhesive. In some embodiments, backing pad 205 includes a tab 207 that extends from one side of backing pad 205. The liner may be set aside for use during a later operation.

FIG. 2B depicts a view of the electrode shown in FIG. 2A following the placement of a conductive layer 210 on the top surface of backing pad 205, according to some embodiments. Conductive layer 210 may be centered horizontally on backing pad 205. In some examples, conductive layer 210 includes a tab 212 extending from one side of conductive layer 210 and tab 212 extends into at least a portion of tab 207 of backing pad 205. Conductive layer 210 may be aligned such that there is about ⅛ inch, about ¼ inch, or about ½ inch of space between a bottom of tab 207 and a bottom of tab 212.

In some embodiments, conductive layer 210 is attached to backing pad 205 via the adhesive top surface of backing pad 205. Conductive layer 210 may be pressed or rolled onto backing pad 205 without creasing any part of conductive layer 210. In some embodiments, a bottom surface of conductive layer 210 includes an adhesive for attaching to the top surface of backing pad 205.

FIG. 2C depicts a view of the electrode shown in FIG. 2B following the placement of a first bonding layer 215, according to some embodiments. First bonding layer 215 may be a double-sided tape or other pressure sensitive adhesive that is pressed onto conductive layer 210. In some embodiments, first bonding layer 215 is aligned over tab 212 of conductive layer 210. In some embodiments, first bonding layer 215 includes a liner on each of a top and bottom surface to protect adhesive layers on each of the top and bottom surfaces. Thus, a liner on the bottom surface of first bonding layer 215 may first be removed before pressing first bonding layer 215 onto conductive layer 210. Another liner 220 may remain on a top surface of first bonding layer 215.

According to some embodiments, the liner removed from backing pad 205 may be placed over first bonding layer 215 to allow for more even pressure to be applied across the top surface of first bonding layer 215. In one example, a wire-to-plate press fixture may be used to apply up to 90 psi of pressure over first bonding layer 215. First bonding layer 215 may have a length between about 1 inch and about 1.5 inches and a width between about 0.75 inch and about 1.25 inches.

FIG. 2D depicts a view of the electrode shown in FIG. 2C following the removal of liner 220, according to some embodiments. Removing liner 220 exposes a top surface 225 of first bonding layer 215. According to some embodiments, top surface 225 is adhesive and may be the same adhesive as used on the bottom surface of first bonding layer 215.

FIG. 2E depicts a view of the electrode shown in FIG. 2D following the attachment of a leadwire 230 to first bonding layer 215, according to some embodiments. The insulation around a distal end of leadwire 230 may be removed to expose stripped end length 235. For example, around one inch of insulation may be removed from the end of leadwire 230 to expose around an inch of stripped end length 235.

As discussed above, stripped end length 235 may include a plurality of extensions (e.g., wires, strands, or filaments) that make up the conductive portion of leadwire 230. The plurality of extensions may be metal or conductive metal coated carbon fibers, such as nickel plated carbon wire fiber. Additionally, stripped end length 235 may include any number of copper strands, such as around four copper strands.

According to an embodiment, leadwire 230 is first aligned over or near a center line of first bonding layer 215. In some examples, a top edge of the insulating portion of leadwire 230 (where stripped end length 235 begins) is aligned to be between about 0.25 inch and about 0.5 inch up from the bottom edge of first bonding layer 215. According to some embodiments, leadwire 230 may be rotated such that the side of stripped end length 235 that includes the copper strands faces away from top surface 225 of first bonding layer 215. Leadwire 230 may then be pressed down onto top surface 225 to bond leadwire 230 to top surface 225 of first bonding layer 215. In some examples, leadwire 230 may also bond to a top surface of the portion of backing pad 205 that extends below first bonding layer 215.

Once leadwire 230 has been bonded to at least first bonding layer 215, stripped end length 235 may be anchored to top surface 225 of first bonding layer 215 by applying an acute pressure on a portion of stripped end length 235 that directly emerges from the insulated end of leadwire 230. FIG. 2E′ illustrates how pressure may be applied, as indicated by the arrow, to stripped end length 235 to anchor it directly adjacent to the insulator around the end of leadwire 230. In some examples, no space exists between stripped end length 235 and first bonding layer 215 as it leaves the insulated end of leadwire 230. For example, the acute pressure applied to stripped end length 235 may curve and bend stripped end length 235 along the insulated end of leadwire 230 until it contacts top surface 225 of first bonding layer 215.

From the anchored point of stripped end length 235, any number of the extensions of stripped end length 235 may be flared outward into a fanned configuration as illustrated in FIG. 2E. The flared extensions may be pressed onto top surface 225 of first bonding layer 215 to bond them in the fanned arrangement. According to some embodiments, the extensions may be flared outward such that a flared top edge of stripped end length 235 extends to nearly an entire width of first bonding layer 215. A distance between the flared top edge of stripped end length 235 and the top edge of first bonding layer 215 may be between about ⅛ inch and ½ inch.

FIG. 2F depicts a view of the electrode shown in FIG. 2E following the placement of a second bonding layer 240 directly on first bonding layer 215 and stripped end length 235, according to some embodiments. Second bonding layer 240 may have substantially the same size as first bonding layer 215. In some embodiments, second bonding layer 240 has the same material composition and structure as first bonding layer 215. In one example, second bonding layer 240 is a double-sided tape or single-sided tape with a bottom surface having an adhesive to bond with top surface 225 of first bonding layer 215 and to bond with stripped end length 235. In some examples, the bottom surface of second bonding layer 240 also covers at least a portion of the insulated end of leadwire 230. A liner layer may be first removed from the bottom surface of second bonding layer 240 before pressing second bonding layer 240 over first bonding layer 215.

According to some embodiments, firm pressure is applied to the top surface of second bonding layer 240 in order to conform the bottom surface of second bonding layer 240 around at least the various flared extensions of stripped end length 235. In some embodiments, pressure is applied to second bonding layer 240 to remove as much space as possible between second bonding layer 240 and first bonding layer 215 and between second bonding layer 240 and stripped end length 235. In some examples, another liner is removed from a top surface of second bonding layer 240 and the liner from backing pad 205 is again laid over second bonding layer 240 to apply equal pressure across second bonding layer 240. Afterwards, the liner removed from the top surface of second bonding layer 240 may be laid over second bonding layer 240 to apply acute pressure around areas like the edges of second bonding layer 240 and around the various flared extensions of stripped end length 235. FIG. 2F′ illustrates how second bonding layer 240 may conform tightly around stripped end length 235. As discussed above, the adhesiveness of each of first bonding layer 215 and second bonding layer 240 confers enhanced rigidity against shear forces that may otherwise jostle the coupling between first bonding layer 215, stripped end length 235, and second bonding layer 240.

FIG. 2G depicts a view of the electrode shown in FIG. 2F following the placement of an insulator layer 245, according to some embodiments. A bottom surface of insulator layer 245 may be adhesive such that insulator layer 245 bonds to exposed top surfaces of various electrode components, such as backing pad 205, conductive layer 210, and second bonding layer 240. In some examples, insulator layer 245 also attaches to a portion of leadwire 230 extending below the bottom edge of second bonding layer 240.

According to some embodiments, insulator layer 245 covers an entirety of second bonding layer 240 and a lower portion of both conductive layer 210 and backing pad 205. Insulator layer 245 may have the same general shape as the lower portion of backing pad 205 such that a lower edge of insulator layer 245 aligns along a lower edge of backing pad 205. According to some embodiments, insulator layer 245 has an area that is less than half or less than ⅓ of an area of backing pad 205. A majority of conductive layer 210 extends above a top edge of insulator layer 245, such as at least 75%, at least 80%, at least 85%, or at least 90% of conductive layer 210 remains exposed above the top edge of insulator layer 245. In some examples, a distance between a top edge of insulator layer 245 and a top edge of second bonding layer 240 is between about ⅛ inch and ½ inch.

FIG. 2H depicts a view of the electrode shown in FIG. 2G following the placement of a gel layer 250, according to some embodiments. Gel layer 250 may cover the entire exposed top layer of conductive layer 210 (e.g., the entire portion of conductive layer 210 not covered by insulator layer 245). In some embodiments, gel layer 250 is disposed on a top surface of conductive layer 210 and on a top surface of insulator layer 245. Gel layer 250 may extend beyond a perimeter of conductive layer 210 by between ⅛ inch and ½ inch.

FIG. 2I depicts a view of the electrode shown in FIG. 2H following the placement of a tab 255, according to some embodiments. Tab 255 may include a bottom adhesive surface to attach tab 255 to a top surface of insulator layer 245. In some embodiments, tab 255 is placed at a bottom edge of insulator layer 245, such that at least a portion of tab 255 extends beyond the bottom edge of insulator layer 245. According to some embodiments, a top surface of tab 255 does not include any adhesive such that it will not stick to the skin of a subject.

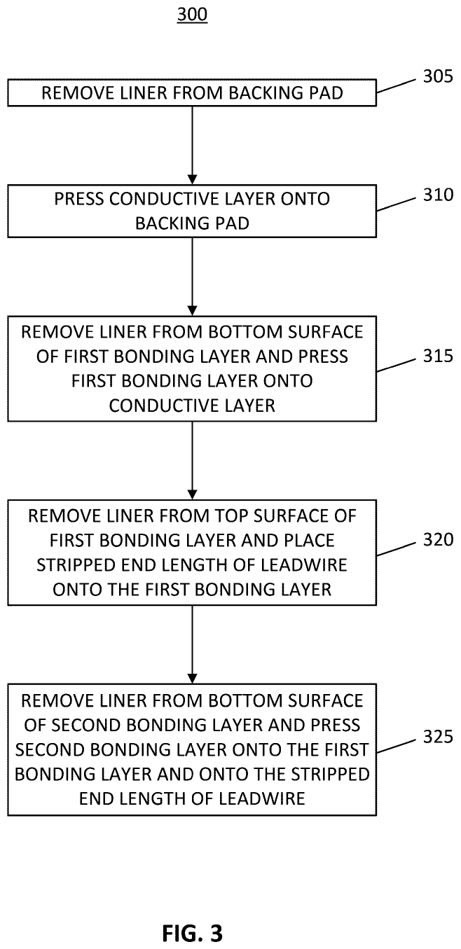

FIG. 3 is a flow chart of a method 300 for forming an electrode, according to an embodiment. Various operations of method 300 may be illustrated in FIGS. 2A-2I. However, the correlation of the various operations of method 300 to the specific components illustrated in the aforementioned figures is not intended to imply any structural and/or use limitations. Rather, the aforementioned figures provide one example embodiment of method 300. Other operations may be performed before, during, or after any of the operations of method 300. Some of the operations of method 300 may be performed in a different order than the illustrated order.

Method 300 begins with operation 305 where a backing pad (e.g., backing pad 105 of FIG. 1A) is provided and a liner is removed from a top surface of the backing pad. The removal of the liner may reveal an adhesive on the top surface of the backing pad. The bottom surface of the backing pad may not include any adhesive. As discussed above, the backing pad may have any shape, such as a circular shape or a rectangular shape.

Method 300 continues with operation 310 where a conductive layer (e.g., conductive layer 110 of FIG. 1A) is pressed onto the top surface of the backing pad. The conductive layer may be generally centered over backing pad 205 (e.g., having a central axis within 1 mm of a central axis of backing pad 205). In some examples, the conductive layer includes a tab extending from one side of the conductive layer and this tab extends into at least a portion of a protrusion extending from one side of the backing pad.

In some embodiments, the conductive layer is attached to the backing pad via the adhesive top surface of the backing pad. The conductive layer may be pressed or rolled onto the backing pad without creasing any part of the conductive layer. In some embodiments, a bottom surface of the conductive layer includes an adhesive for attaching to the top surface of the backing pad.

Method 300 continues with operation 315 where a liner is removed from a bottom surface of a first bonding layer (e.g., first bonding layer 125 of FIG. 1A) and the bottom surface of the first bonding layer is subsequently attached to a portion of the conductive layer. According to some embodiments, the first bonding layer is aligned over the tab that extends from one side of the conductive layer and is attached to a top surface of this tab. The liner may be peeled away from the bottom surface of the first bonding layer to reveal an adhesive on the bottom surface. Accordingly, the first bonding layer may be a conductive double-sided tape. According to some embodiments, the liner removed from the backing pad may be placed over the first bonding layer to allow for more even pressure to be applied across the top surface of the first bonding layer. In some embodiments, the first bonding layer does not have a liner and is attached directly to the top surface of the conductive layer without the need to remove any liner layer.

Method 300 continues with operation 320 where a liner is removed from a top surface of the first bonding layer and a stripped end length of leadwire (e.g., stripped end length 120 of FIG. 1A) is placed on the top surface of the first bonding layer. Removing the liner from the top surface of the first bonding layer may reveal an adhesive surface on the first bonding layer. According to some embodiments, about 1 inch of insulation may be removed from the end of the leadwire to expose the conductive stripped end length of wire. The stripped end length may include a plurality of conductive strands, such as carbon filaments coating in nickel. In some embodiments, the stripped end length also includes a few (e.g., four) copper wires that are larger than each of the carbon filaments.

According to some embodiments, the leadwire is placed at one edge of the top surface of the first bonding layer such that a portion of the insulated end of the leadwire is attached to the top surface of the first bonding layer. An acute pressure may be applied at the point where the stripped end length exits from the insulated portion of the leadwire in order to press the stripped end length onto the top surface of the first bonding layer. This action may provide an anchor point for the stripped end length as it leaves the insulated leadwire. In some examples, no space exists between the stripped end length and the top surface of the first bonding layer and no space exists between the stripped end length and the edge of the insulated portion of the leadwire. The acute pressure may be applied using any suitable tool, such as tweezers.

Either before or after the stripped end length has been anchored to the top surface of the first bonding layer, one or more of the extensions or groups of extensions of the stripped end length are spread out into a fanned arrangement across the top surface of the first bonding layer, according to some embodiments. The flared extensions may be pressed onto the top surface of the first bonding layer to bond them in the fanned arrangement. According to some embodiments, the extensions may be flared outward such that a flared top edge of the stripped end length extends to nearly an entire width of the first bonding layer. A distance between the flared top edge of the stripped end length and the top edge of the first bonding layer may be between about ⅛ inch and ½ inch, and a distance between the sides of the first bonding layer and the farthest lateral extensions of the stripped end length may be between about 1/32 inch and about ⅛ inch.

Method 300 continues with operation 325 where a liner is removed from the bottom surface of a second bonding layer (e.g., second bonding layer 130 of FIG. 1A) and the bottom surface of the second bonding layer is pressed onto the top surface of the first bonding layer and onto the stripped end length. According to some embodiments, removing the liner from the bottom surface of the second bonding layer exposes an adhesive surface on the bottom of the second bonding layer.

The second bonding layer may have substantially the same size as the first bonding layer. According to some embodiments, the second bonding layer is aligned directly over the first bonding layer such that the perimeter of the second bonding layer substantially aligns with the perimeter of the first bonding layer as they are pressed together. In one example, the second bonding layer is a double-sided tape or single-sided tape with a bottom surface having an adhesive to bond with the top surface of the first bonding layer and to bond with the stripped end length. In some examples, the bottom surface of the second bonding layer also covers at least a portion of the insulated end of the leadwire.

According to some embodiments, firm pressure is applied to the top surface of the second bonding layer in order to conform the bottom surface of the second bonding layer around at least the various fanned extensions of the stripped end length. In some embodiments, pressure is applied to the second bonding layer to remove as much space as possible between the second bonding layer and the first bonding layer and between the second bonding layer and the stripped end length. In some examples, the liner from the backing pad is laid over the second bonding layer to apply equal pressure across the second bonding layer. Afterwards, the liner removed from the top surface of the second bonding layer may be laid over the second bonding layer to apply acute pressure around areas like the edges of the second bonding layer and around the various fanned extensions of the stripped end length.

Any number of other operations may be performed to complete the fabrication of the electrode. For example, an insulator layer (e.g., insulator layer 135 of FIG. 1A) may be attached over the bottom portion of the backing pad and over the second bonding layer. Following the insulator layer, a conductive gel layer (e.g., conductive gel layer 140 of FIG. 1A) may be placed over the exposed portion of the conductive layer and over a portion of the insulator layer. The conductive gel layer may cover at least an entire exposed portion of the conductive layer (e.g., the portion of the conductive layer not under the insulator layer). In some examples, at least one tab (e.g., tab 145 of FIG. 1A) is attached to the top surface of the insulator layer along a bottom edge of the insulator layer.

### Labeling and Packaging

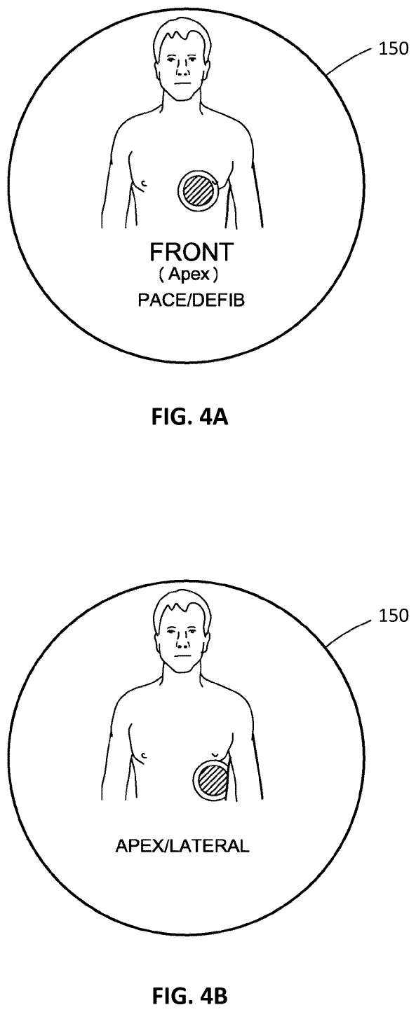

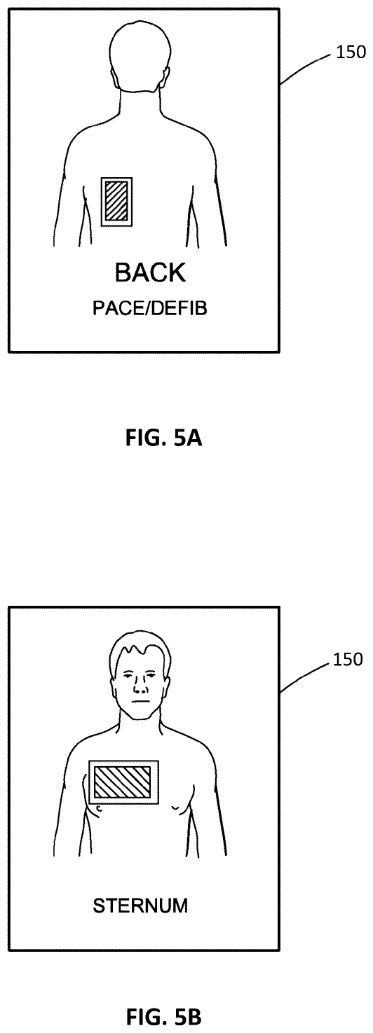

FIG. 4A depicts an example label 150 with instructions printed on the label to direct a user (e.g., the subject or a health care technician) to place electrode 100 in an anterior position on the subject's chest proximate to the subject's heart. FIG. 4B depicts an example label 150 indicating that electrode 100 is to be placed in a lateral position on the subject. FIG. 5A depicts an example label 150 with instructions printed on the label to direct a user to place electrode 100 in a posterior position on the subject's back proximate to the subject's heart. FIG. 5B depicts an example label 150 indicating that electrode 100 is to be placed in an anterior position on the subject.

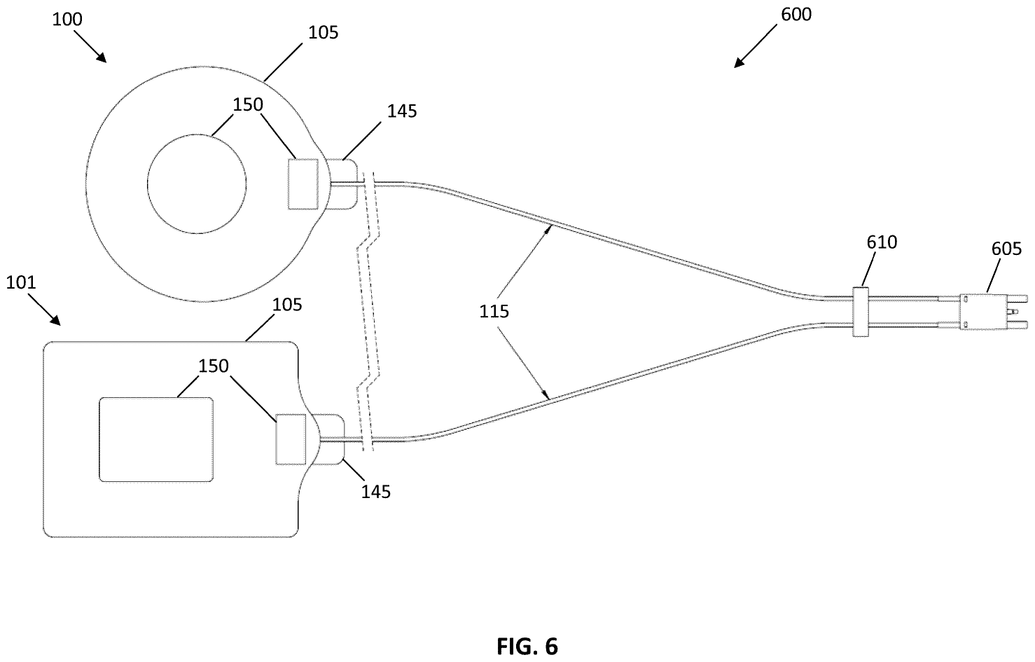

FIG. 6 is a plan view of an electrode system 600 that includes a pair of electrodes 100 and 101. In one embodiment, electrodes 100 and 101 are part of biomedical electrode system 600 that is configured to treat or monitor a subject. For example, the subject can wear each of electrodes 100 and 101. Leadwires 115 run from each electrode to a defibrillating, pacing, monitoring, or control unit (not shown in FIG. 6), which may include a power source, to control the operation of electrodes 100/101 or to process electrical signals (e.g., EKG, EEG) detected by electrodes 100/101. The two electrodes 100/101, leadwires 115, and the subject's body form part of a circuit so that electrical energy or signals may be passed to or from the subject via electrodes 100/101. The control unit, power supply or other devices can also be included in this circuit.

Electrodes 100/101 may have different shapes for placement on different areas of the subject's body, e.g., anterior, posterior, or lateral. With reference to FIG. 6, electrode 100 may be substantially circular for anterior placement on the subject's chest, for example proximate to the subject's heart. Another electrode 101 may be substantially rectangular for posterior placement on the subject's back, for example also proximate to the subject's heart. Electrodes 100/101 may be used to defibrillate, pace, cardiovert, or monitor the heart.

In one embodiment, at least one connector 605 receives at least one leadwire 115. For example, a first end of connector 605 can receive a terminal end of leadwire 115 that is distal from stripped end length 120. A second end of connector 605 can interface with at least one defibrillator, power supply, monitor, pacing unit, or control system. Connector 605 can secure leadwires 115, providing strain relief. In one embodiment, leadwires 115 are connected to connector 605 by a press fit, or anchored by cold-forming dimples in connector 605. Connector 605 may be made of a substantially rigid plastic or other material. In one embodiment, connector 605 receives a first leadwire 115 that is coupled with electrode 100, and connector 605 receives a second leadwire 115 that is coupled with electrode 101. An indication of which leadwire 115 couples with which electrode can be printed on leadwire 115 or on a label attached to leadwire 115, for example near the point of connection with connector 605.

In one embodiment, biomedical electrode system 600 includes at least one containment element 610. At least one leadwire 115 can pass through containment element 610 between connector 605 and electrode 100. For example, containment element 610 may be located within three inches, measuring along the length of leadwire 115, from connector 605. Containment element 610 can secure leadwires 115, maintain the integrity of the connection between leadwires 115 and connector 605, and provide strain relief. Containment element 610 can be made of a substantially rigid plastic or other material. In one embodiment, containment element 610 is an integral unit with one through-hole for each leadwire 115. Containment element 610 can have a variety of shapes, for example, rectangular, square, cubed, spherical, or elongated and curved, having a profile similar to a prolate spheroid.

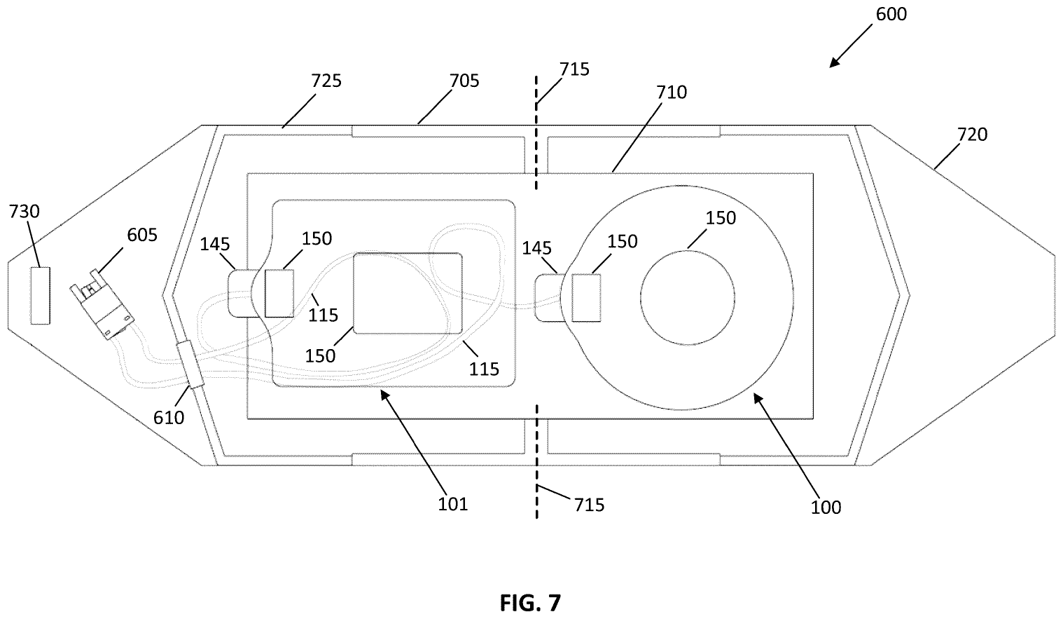

FIG. 7 is a plan view depicting biomedical electrode system 600. In one embodiment, electrode system 600 is included in package 705, such as a book or pouch. Package 705 can be made of plastic, and can contain at least two electrodes 100/101 that have been assembled and are ready to be positioned on a subject. In one embodiment, package 705 has sleeves, with one or more electrodes disposed in each sleeve. Package 705 may also include layer 710, such as a polyester film. The adhesive bottom surfaces of electrode components can attach to a coated surface of layer 710 when disposed in package 705. In one embodiment, package 705 and layer 710 can fold, substantially in half along axis 715. Flaps 720 can close over package 705 when package 705 is in a folded configuration.

Edges 725 can assist in the closing of package 705 and maintain the integrity of package 705 in a closed position. Edges 725 can be reinforced. In one embodiment, containment element 610 is located at edges 725 with leadwires 115 passing through containment element 610. In one embodiment, containment element 610 seals leadwires 115 to package 705. This seal can be airtight. Connector 605 can be located in the part of package 705 that includes flap 720, which may include a separate pouch for connector 605, segregating connector 605 from the other components of electrode 100/101 and biomedical electrode system 600. This can prevent sharp or hard parts of connector 605 from damaging other components. The leads of connector 605 can be secured in element 730 to secure connector 605 and other electrode system components. Electrodes 100/101 or biomedical electrode system 600 can be packaged in packages having different configurations than that of package 705. For example, a package can include a single bag, pouch, sleeve, or pocket for all biomedical electrode system 600 components.

The foregoing description is illustrative and not limiting, having been presented by way of example. In particular, although many of the examples presented herein involve specific combinations of method acts or system elements, those acts and those elements may be combined in other ways. Acts, elements and features discussed only in connection with one embodiment are not intended to be excluded from a similar role in other embodiments.

Note that in FIGS. 1 through 7, the enumerated items are shown as individual elements. In actual implementations of the systems and methods described herein, however, they may be part of or inseparable components of other elements.

Any references to embodiments or elements or acts of the systems and methods herein referred to in the singular may also embrace embodiments including a plurality of these elements, and any references in plural to any embodiment or element or act herein may also embrace embodiments including only a single element. References in the singular or plural form are not intended to limit the presently disclosed systems or methods, their components, acts, or elements to single or plural configurations. References to any act or element being based on any information, act or element may include embodiments where the act or element is based at least in part on any information, act, or element.

Any embodiment disclosed herein may be combined with any other embodiment, and references to “an embodiment,” “some embodiments,” “an alternate embodiment,” “various embodiments,” “one embodiment” or the like are not necessarily mutually exclusive and are intended to indicate that a particular feature, structure, or characteristic described in connection with the embodiment may be included in at least one embodiment. Such terms as used herein are not necessarily all referring to the same embodiment. Any embodiment may be combined with any other embodiment, inclusively or exclusively, in any manner consistent with the aspects and embodiments disclosed herein.

References to “or” may be construed as inclusive so that any terms described using “or” may indicate any of a single, more than one, and all of the described terms. Embodiments, acts, or elements are not essential unless recited as such.

One skilled in the art will realize the systems and methods described herein may be embodied in other specific forms without departing from the characteristics thereof. For example, electrode components may have shapes other than circular and rectangular. Electrode components can be circular, elliptical, quadrilateral, square, or other designs, and can have different sizes for larger (e.g. adult or obese) or smaller (child, pediatric, or neonatal) subjects. Electrodes configured for placement on a particular part of the subject's anatomy (e.g., chest, back, legs, head) can be ergonomically configured to adhere to that anatomical feature. A plurality of electrodes can be placed on one region of the subject, e.g., two electrodes can be placed on the subject's chest, back, or lateral portion, with at least one other electrode placed at another location on the subject. Electrodes can be concave or convex. Electrodes can have more fanciful or arbitrary shapes or patterns (e.g, star, unicorn, smiley face, dinosaur, football, baseball, soccer ball, basketball, celebrity, athletic, or cartoon figure) to for example ease the mental anguish of a child with a medical condition who wears the electrode. Further, the adhesive couplings described herein can be permanent or reversible. For example, they can be peeled apart for maintenance or replacement purposes. Other couplings, press fit, mechanical, fasteners, etc can also couple elements together. The couplings can allow electrical currents to pass between the subject and leadwire 115 via a plurality of components (e.g., stripped end length 120, conductive layer 110, and conductive gel layer 140) directly or via intervening components. Further, electrode components depicted in dashed lines are part of the electrode and merely may not be entirely visible from the perspective of the associated drawings.

The foregoing embodiments are illustrative rather than limiting of the described systems and methods. Scope of the systems and methods described herein is thus indicated by the appended claims, rather than the foregoing description, and changes that come within the meaning and range of equivalency of the claims are embraced therein.

1. A radio transmissive electrode for providing electrotherapeutic defibrillation to a patient, the electrode comprising:
a backing pad;
a conductive layer disposed upon the backing pad;
a bonding layer disposed upon the conductive layer; and
a leadwire comprising a stripped end length, at least a portion of the stripped end length being disposed upon the bonding layer, such that the bonding layer is interposed between the conductive layer and the stripped end length.

2. The electrode of claim 1, wherein the bonding layer is a first bonding layer and the electrode further comprises a second bonding layer disposed on the first bonding layer and disposed on the stripped end length of the leadwire, such that the stripped end length of the leadwire is interposed between the first bonding layer and the second bonding layer.

3. The electrode of claim 2, further comprising an insulator layer disposed on the second bonding layer.

4. The electrode of claim 3, wherein the insulator layer is disposed on at least a portion of the conductive layer.

5. The electrode of claim 3, wherein a surface area of the insulator layer is less than half of a surface area of the backing pad.

6. The electrode of claim 3, further comprising a gel layer disposed on at least a portion of the conductive layer.

7. The electrode of claim 6, wherein the gel layer is disposed on at least a portion of the insulator layer.

8. The electrode of claim 6, further comprising a tab coupled to a lower surface of the gel layer and protruding from the electrode.

9. The electrode of claim 2, wherein a first surface and an opposite second surface of the first bonding layer comprises an adhesive, and a first surface and an opposite second surface of the second bonding layer comprises an adhesive.

10. The electrode of claim 9, wherein the first surface of the second bonding layer conforms around the stripped end length of the leadwire.

11. The electrode of claim 1, wherein the leadwire includes a conductive carbon element, and wherein the stripped end length includes a plurality of stripped extensions.

12. The electrode of claim 11, wherein the plurality of stripped extensions are in a fanned configuration.

13. The electrode of claim 11, wherein the plurality of stripped extensions are adhesively coupled to the bonding layer.

14. The electrode of claim 1, wherein the electrode is radiolucent.

15. The electrode of claim 1, wherein the electrode is radiotransparent.

16. The electrode of claim 1, wherein the electrode includes at least one of: a therapy electrode, a treatment electrode, a stimulating electrode, and a monitoring electrode; and wherein the electrode is configured to adhere to a subject.

17. The electrode of claim 1, wherein the conductive layer comprises silver (Ag) and silver chloride (AgCl).

18. The electrode of claim 17, wherein the conductive layer comprises more than 50% Ag by weight, and less than 50% AgCl by weight.

19. The electrode of claim 1, wherein the bonding layer is a conductive double-sided tape.

20. The electrode of claim 1, wherein the bonding layer comprises a plurality of sublayers.

21. The electrode of claim 20, wherein the plurality of sublayers comprises a first adhesive layer, a carrier layer on the first adhesive layer, and a second adhesive layer on the carrier layer.

22. The electrode of claim 21, wherein the carrier layer comprises a conductive nonwoven material.

23. The electrode of claim 1, wherein a thickness of the bonding layer is between about 110 micrometers and about 130 micrometers.

24. The electrode of claim 1, wherein the bonding layer is radio transmissive.

25. The electrode of claim 1, wherein the bonding layer is mechanically rigid.

26. The electrode of claim 1, wherein the bonding layer is a conductive, double-sided adhesive that is radio transmissive and mechanically rigid.

27-60. (canceled)
